(19) United States
(12) Patent Application Publication
(10) Pub. No.: US 2026/0131129 A1
(43) Pub. Date: May 14, 2026

(54) Left Atrium Unloading Device

(71) Applicant: University of Denver, Denver, CO (US)

(72) Inventor: Ali Nejatbakhsh Azadani, Greenwood Village, CO (US)

(21) Appl. No.: 19/386,734

(22) Filed: Nov. 12, 2025

Related U.S. Application Data
(60) Provisional application No. 63/719,471, filed on Nov. 12, 2024.

(51) Int. Cl. A61M 60/165; A61M 60/232; A61M 60/416; A61M 60/812; A61M 60/814; A61M 60/82; A61M 60/871

(52) U.S. Cl. CPC A61M 60/165; A61M 60/232; A61M 60/416; A61M 60/812; A61M 60/814; A61M 60/82; A61M 60/871; A61M 2205/04; A61M 2205/103; A61M 2205/3334; A61M 2210/125; A61M 2210/127

(57) ABSTRACT

Left atrium unloading devices and systems, including an inflow section configured to receive blood from a left atrium; an impeller having multiple blades arranged move the blood; a motor to rotate the impeller; a volute; and an outflow section configured to direct the blood from the volute to a descending aorta.

### REFERENCE TO RELATED APPLICATIONS

The present invention claims the benefit of U.S. Provisional Ser. No. 63/719,471 , filed on Nov. 12, 2024, and titled “Left Atrium Unloading Device,” which is incorporated hereby in its entirety by reference.

### SPONSORED RESEARCH AND DEVELOPMENT

This invention was made with government support under Grant No. IA-0000063101 awarded by the State of Colorado, Office of Economic Development & International Trade (OEDIT) Advanced Industries Proof of Concept (AI-POC) Grant. The government has certain rights in the invention.

### FIELD OF THE DISCLOSURE

The present disclosure relates to mechanical circulatory support (MCS) devices. In particular, but not by way of limitation, the present disclosure relates to implantable left atrium unloading devices for supporting the functioning of the heart for patients with heart failure with preserved ejection fraction.

### DESCRIPTION OF RELATED ART

Heart failure represents a complex cardiovascular syndrome affecting millions of individuals worldwide, with the condition broadly categorized based on left ventricular ejection fraction measurements. Heart failure with preserved ejection fraction (HFpEF) constitutes a substantial portion of heart failure cases, characterized by normal or near-normal ejection fraction values while patients experience typical heart failure symptoms including exertional dyspnea, exercise intolerance, and fluid retention.

The occurrence of HFpEF continues to increase globally, affecting approximately 3.5 million individuals in the United States and nearly 35 million people globally. HFpEF significantly diminishes quality of life, particularly in patients with advanced disease, and contributes substantially to the healthcare burden, accounting for approximately 700,000 hospitalizations each year in the United States. This condition is further characterized by a high rate of comorbidities and an associated mortality rate of approximately 25 percent. As the population continues to age, the prevalence of HFpEF is projected to increase by nearly 50 percent over the next decade, with the annual economic burden in the United States expected to rise to approximately $35 billion.

### BRIEF DESCRIPTION OF DRAWINGS

FIG. 1 shows an illustrative Computed Tomography Angiography (CTA) image of a heart and portions of a circulatory system

FIG. 2 shows an illustrative CTA image of a heart and portions of a circulatory system with placement of a Left Atrium Unloading Device (LAUD) and outflow cannula, in accordance with various embodiments of the present disclosure.

FIG. 3 shows an illustrative exploded perspective view of a LAUD, in accordance with various embodiments of the present disclosure.

FIG. 4 shows an illustrative exploded side view of a LAUD, in accordance with various embodiments of the present disclosure.

FIG. 5 shows an illustrative isometric view of a LAUD, in accordance with various embodiments of the present disclosure.

FIG. 6 shows an illustrative isometric view of an impeller for a LAUD, in accordance with various embodiments of the present disclosure.

FIG. 7 shows an illustrative isometric view of an impeller for a LAUD, in accordance with various embodiments of the present disclosure.

FIG. 8 shows an illustrative graph of impeller efficiency comparison for various designs of a LAUD, in accordance with various embodiments of the present disclosure.

FIG. 9 shows an illustrative graph of head efficiency for various designs of a LAUD, in accordance with various embodiments of the present disclosure.

For simplicity and clarity of illustration, the drawing figures illustrate the general manner of construction, and descriptions and details of well-known features and techniques may be omitted to avoid unnecessarily obscuring the embodiments detailed herein. Additionally, elements in the drawing figures are not necessarily drawn to scale. For example, the dimensions of some of the elements in the figures may be exaggerated relative to other elements to help improve understanding of the described embodiments. The same reference numerals in different figures denote the same elements.

The word “exemplary” is used herein to mean “serving as an example, instance, or illustration.” Any embodiment described herein as “exemplary” is not necessarily to be construed as preferred or advantageous over other embodiments. In the following detailed description, references are made to the accompanying drawings that form a part hereof, and in which are shown by way of illustrations or specific examples. These aspects may be combined, other aspects may be utilized, and structural changes may be made without departing from the present disclosure. Example aspects may be practiced as methods, systems, or apparatuses. The following detailed description is therefore not to be taken in a limiting sense, and the scope of the present disclosure is defined by the appended claims and their equivalents.

### DETAILED DESCRIPTION

The following presents a simplified summary relating to one or more aspects and/or embodiments disclosed herein. As such, the following summary should not be considered an extensive overview relating to all contemplated aspects and/or embodiments, nor should the following summary be regarded to identify key or critical elements relating to all contemplated aspects and/or embodiments or to delineate the scope associated with any particular aspect and/or embodiment. Accordingly, the following summary has the sole purpose to present certain concepts relating to one or more aspects and/or embodiments relating to the mechanisms disclosed herein in a simplified form to precede the detailed description presented below.

These and other features, and characteristics of the present disclosure, as well as the methods of operation and functions of the related elements of structure and the combination of parts and economies of manufacture, will become more apparent upon consideration of the following description and the appended claims with reference to the accompanying drawings, all of which form a part of this specification, where like reference numerals designate corresponding parts in the various figures. It is to be expressly understood, however, that the drawings are for the purpose of illustration and description only and are not intended as a definition of the limits of the invention. As used in the specification and in the claims, the singular form of “a,” “an,” and “the” include plural referents unless the context clearly dictates otherwise.

In the description herein, various ranges and/or numerical limitations can be expressly stated below. It should be recognized that unless stated otherwise, it is intended that endpoints are to be interchangeable. Further, any ranges include iterative ranges of like magnitude falling within the expressly stated ranges or limitations.

Furthermore, various modifications can be made within the scope of the invention as herein intended, and embodiments of the invention can include combinations of features other than those expressly claimed. In particular, flow arrangements other than those expressly described herein are within the scope of the invention.

Regarding claim transitional terms or phrases, the transitional term “comprising”, which is synonymous with “including,” “containing,” “having” or “characterized by,” is inclusive or open-ended and does not exclude additional, unrecited elements or method steps. The transitional phrase “consisting of” excludes any element, step, or ingredient not specified in the claim. The transitional phrase “consisting essentially of” limits the scope of a claim to the specified materials or steps and those that do not materially affect the basic and novel characteristic(s) of the claimed invention. A “consisting essentially of” claim occupies a middle ground between closed claims that are written in a “consisting of” format and fully open claims that are drafted in a “comprising” format. Absent an indication to the contrary, when describing a compound or composition “consisting essentially of” is not to be construed as “comprising,” but is intended to describe the recited component that includes materials which do not significantly alter composition or method to which the term is applied.

Within this specification, use of “comprising” or an equivalent expression contemplates the use of the phrase “consisting essentially of,” “consists essentially of,” or equivalent expressions as alternative embodiments to the open-ended expression. Additionally, use of “comprising” or an equivalent expression or use of “consisting essentially of” in the specification contemplates the use of the phrase “consisting of,” “consists of,” or equivalent expressions as an alternative to the open-ended expression or middle ground expression, respectively. For example, “comprising” should be understood to include “consisting essentially of,” and “consisting of” as alternative embodiments for the aspect, features, and/or elements presented in the specification unless specifically indicated otherwise.

While compositions and methods are described in terms of “comprising” various components or steps, the compositions and methods can also “consist essentially of” or “consist of” the various components or steps.

Current therapeutic approaches for HFpEF patients face considerable limitations. Pharmacological treatments present implementation challenges due to factors including complexity, patient tolerance, and system-related issues. These challenges contribute to high rehospitalization rates, with approximately 29 percent of patients requiring readmission within 60 to 90 days of discharge. Traditional heart failure medications that have proven effective for heart failure with reduced ejection fraction have not demonstrated comparable efficacy in HFpEF patients.

The pathophysiology of HFpEF involves impaired left ventricular relaxation and reduced left atrial compliance, leading to elevated left atrial pressure and pulmonary capillary wedge pressure, particularly during exercise. These hemodynamic changes directly correlate with the exercise intolerance and symptoms experienced by HFpEF patients. Left atrial size and function have emerged as potential therapeutic targets, as larger left atrial dimensions correlate with increased hospitalization risk and adverse outcomes.

There exists a need for mechanical circulatory support devices specifically designed and optimized for the unique hemodynamic requirements of HFpEF patients, particularly devices capable of reducing left atrial pressure while enhancing cardiac output at the lower flow rates appropriate for this patient population.

An improved treatment for HFpEF may reduce left atrial (LA) pressure, increase cardiac output (CO), or both. Patients with end-stage heart failure with reduced ejection fraction (HFrEF) have benefited from durable mechanical circulatory support (MCS) devices, which have been shown to improve survival and quality of life when used as a bridge to transplantation or destination therapy. However, despite these advances, comparable MCS devices have not been widely adopted or recommended by the medical community for use in HFpEF patients. In fact, existing MCS devices designed for higher cardiac output scenarios are not well-suited for HFpEF patients, who typically require lower flow rates and present different hemodynamic profiles. The anatomical characteristics of HFpEF patients, including smaller and thicker left ventricular chambers, present additional challenges for conventional device applications. There are also regulatory challenges associated with prior devices, such as the recall of the axial pump-based Medtronic HeartWare™ Ventricular Assist Device (HVAD) system, as documented by the U.S. Food and Drug Administration.

Moreover, device-based treatment alternatives for HFpEF remain extremely limited. As described in prior publications, few systems have been developed or provided improvements for the unique pathophysiology of HFpEF, which may be characterized by diastolic dysfunction, impaired ventricular relaxation, and elevated filling pressures despite a normal ejection fraction. Accordingly, there exists a need for improved systems, devices, and methods specifically designed to treat HFpEF patients by targeting hemodynamic parameters such as left atrial pressure and cardiac output in a manner that is less invasive and compatible with the circulatory dynamics of the HFpEF patient population.

There is also demand for new methods and MCS devices having improvements (e.g., improved benefits, better technology, lower costs, etc.). This demand is expanding due to several factors. For example, there is a growing population of heart failure patients The global prevalence of heart failure continues to rise due to aging populations and improved survival rates from cardiovascular diseases. MCS devices, particularly for patients who are not candidates for heart transplants, are increasingly important for long-term circulatory support. Further, there is a growing availability of technological innovations. Innovations such as fully implantable systems, magnetically levitated pumps, and transcutaneous energy transfer systems are creating new opportunities for improved patient outcomes. Such advancements are reducing complications that can include infection, thrombosis, and bleeding, thereby making MCS devices more appealing to both clinicians and investors. In addition, there are unmet clinical needs because many patients with advanced heart failure, including those with heart failure with preserved ejection fraction (HFpEF), have limited treatment options. New or improved MCS devices can address this need, in particular if they function as alternatives to, or bridges to, heart transplants. Further, the demand for new or improved MCS devices is also driven by economic considerations. For example, major medical device companies are actively acquiring smaller firms with innovative MCS technologies, indicating strong market interest.

Embodiments of the present disclosure advantageously include a MCS device for use in the HFpEF patient population. Embodiments disclosed herein seek to mitigate, alleviate or eliminate one or more deficiencies, disadvantages or issues in the art, such as the above-identified, singly or in any combination by providing devices, systems, and methods according to the present disclosure. Various embodiments include a Left Atrium Unloading Device (LAUD) specifically designed to address challenges in the HFpEF patient population.

Some embodiments disclosed herein provide a device (also referred to as a LAUD) that is a long-term circulatory support system that can reduce left atrium (LA) pressure and improve cardiac output by providing left atrium to aortic mechanical circulatory support.

Various embodiments disclosed herein include a left atrium unloading device, including: an inflow section configured to receive blood from a left atrium; an impeller having multiple blades arranged move the blood; a motor to rotate the impeller; a volute; and an outflow section configured to direct the blood from the volute to a descending aorta.

In some aspects, the impeller includes four blades. In various aspects, the impeller includes six blades.

In some embodiments, the left atrium unloading device is configured to drain blood from a left atrium to a descending aorta at flow rates between about 1.0 and about 2.5 liters per minute.

In some aspects, the impeller includes an impeller eye located at a center of the impeller forming an inlet region where blood enters the impeller.

In various aspects, the volute includes a spiral-shaped chamber configured to convert rotational energy into fluid pressure.

In various embodiments, the device includes bearings supporting a rotating shaft. In some aspects, the bearings include magnetic bearing systems. In some embodiments, the device includes a power supply connector attached to the motor for electrical power delivery.

In some aspects, the device includes an inflow cannula attached to the left atrium and an outflow cannula attached to the descending aorta.

Various embodiments disclosed herein include a method of treating heart failure with preserved ejection fraction, including: implanting a left atrium unloading device within a chest cavity of a patient, the left atrium unloading device including: an impeller; an inflow section configured to receive blood from a left atrium of the patient; and an outflow section configured to transfer blood to a descending aorta of the patient, where the left atrium unloading device is configured to rotate the impeller to remove blood from the left atrium to the descending aorta.

In some aspects, the impeller includes four blades. In various aspects, the impeller includes six blades. In some embodiments, the blades are curved. In various embodiments, the blades are straight.

Various aspects include where implanting the left atrium unloading device includes positioning the device in the patient through a thoracotomy procedure. Some aspects include where implanting the left atrium unloading device includes positioning the device in the patient through a sternotomy procedure.

Various embodiments disclosed herein include a left atrium unloading device for heart failure with preserved ejection fraction patients, including: an inflow section configured to receive blood from a left atrium; an impeller having multiple blades arranged move the blood; a motor to rotate the impeller; a volute; and an outflow section configured to direct the blood from the volute to a descending aorta.

In some aspects, the left atrium unloading device is configured to drain blood from a left atrium to a descending aorta at flow rates between about 1.0 and about 2.5 liters per minute.

In various aspects, the device of the methods includes a power supply connector attached to the motor for electrical power delivery.

In some embodiments, the device includes an inflow cannula attached to the left atrium and an outflow cannula attached to the descending aorta.

According to some embodiments of the present disclosure, a left atrium unloading device is provided. The device includes a compact centrifugal pump configured to drain blood from a left atrium to a descending aorta at flow rates between 1.0 and 2.5 liters per minute. The centrifugal pump includes an inflow section configured to receive blood from the left atrium, an impeller having multiple blades arranged to impart rotational energy to blood, a volute surrounding the impeller and configured to convert rotational energy into fluid pressure, and an outflow section configured to direct blood to the descending aorta. The device further includes a rotating shaft coupled to the impeller, a motor operatively connected to the rotating shaft to rotate the impeller, and bearings supporting the rotating shaft. A casing encloses the centrifugal pump components and provides structural support.

According to various aspects of the present disclosure, the left atrium unloading device may include one or more of the following features. The impeller may include four blades or six blades arranged circumferentially around a central hub. The blades may be straight or curved and positioned at specific angles to facilitate efficient fluid flow and pressure generation. The impeller may include an impeller eye with a center of the eye diameter located at a center of the impeller diameter, and the eye having openings forming an inlet region where blood enters from an inflow section. The volute may have a spiral-shaped chamber that facilitates conversion of rotational energy from the impeller into fluid pressure. The device may include an inflow cannula for attachment to the left atrium and an outflow cannula for attachment to the descending aorta. The motor may be configured with a power supply connector for electrical power delivery. The device may be configured for implantation in a patient. The centrifugal pump may be optimized for low cardiac output scenarios and configured to reduce left atrial pressure while enhancing cardiac output in patients with heart failure with preserved ejection fraction.

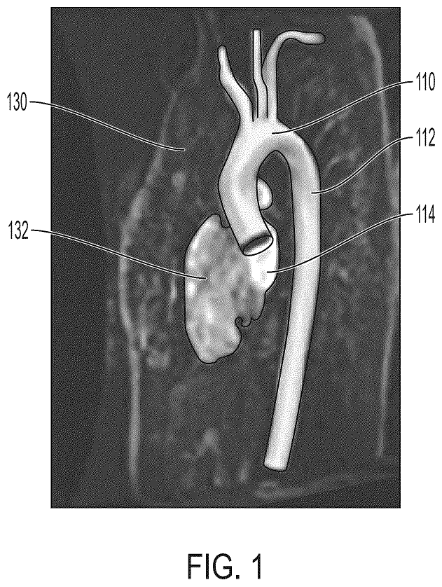

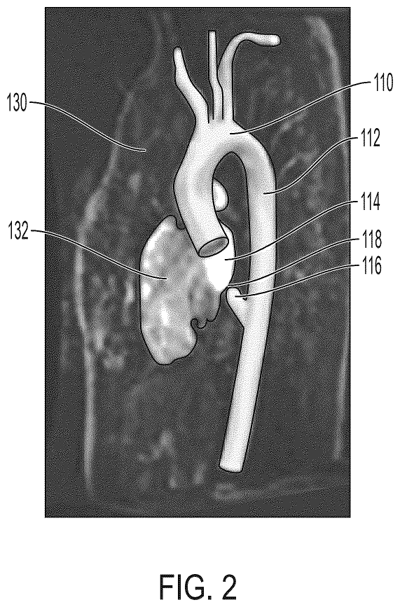

Referring to FIGS. 1-2, illustrative CTA images of a heart and portions of a circulatory system are shown. In various aspects, a LAUD may be positioned within a chest cavity 130 to provide mechanical circulatory support through direct connection between cardiac structures and the arterial system. The chest cavity 130 contains a heart 132 that includes various cardiac chambers and structures involved in blood circulation. An aorta 110 extends from the heart 132 and transitions into a descending aorta 112, forming part of the major arterial pathway for systemic blood circulation. A left atrium 114 forms part of the heart 132 structure and represents the cardiac chamber that receives oxygenated blood from the pulmonary circulation.

In HFpEF patients, the left atrium 114 may experience elevated pressures that contribute to symptoms and clinical complications. In various advantageous aspects, a LAUD addresses this pathophysiology by providing a pathway for blood drainage from the left atrium 114. A LAUD may be anatomically positioned to create a connection between the left atrium 114 and the descending aorta 112. As illustrated in FIGS. 1-2, an outflow cannula 116 extends from the left atrium 114 to the descending aorta 112, establishing a direct pathway for blood flow between these structures. The outflow cannula 116 provides the conduit through which blood may be transported from the left atrium 114 to the descending aorta 112.

An illustrative location of LAUD 118 is indicated along the pathway of the outflow cannula 116, positioned between the left atrium 114 and the descending aorta 112. The location of LAUD 118 demonstrates an illustrative spatial relationship of the device within the chest cavity 130 and its integration with the cardiovascular anatomy. The positioning allows the LAUD to function as an active pumping mechanism within the blood flow pathway established by the outflow cannula 116.

The LAUD may be configured for implantation in a patient through various surgical approaches. In some cases, implantation may be performed via a thoracotomy procedure that provides access to the chest cavity 130 and cardiac structures. Alternative surgical approaches may include traditional sternotomy or other methods that allow for appropriate positioning of the device adjacent to the heart 132 and descending aorta 112. The cannulae and anastomoses may be configured for optimal surgical implementation and hemodynamic performance. For example, the inflow cannula may seat at the LA (e.g., the appendage cuff) with atraumatic fixation and hemostatic sealing; the outflow cannula may terminate in the descending thoracic aorta via an end-to-side anastomosis. In various aspects, lumen diameters may balance low line losses (to preserve pump head and hemocompatibility) with kink resistance and surgical ergonomics; surface finishing and biomaterial coatings may aim to reduce protein adsorption and platelet activation.

LAUDs as disclosed in various embodiments described herein may be constructed from various biocompatible materials selected to optimize performance and safety for long-term implantation. For example, blood-contacting surfaces may be fabricated from biocompatible polymers that provide hemocompatible interfaces and reduce the risk of thrombosis or adverse biological reactions. In some aspects, titanium alloys may be utilized for blood-contacting components due to their favorable biocompatibility profile and corrosion resistance in physiological environments. Structural components of the device may be manufactured from medical-grade stainless steel, which provides mechanical strength and durability for supporting the operational loads of the centrifugal pump assembly. Alternative structural materials may include polyetheretherketone (PEEK), which can offer advantageous properties including radiolucency, chemical resistance, and mechanical characteristics suitable for implantable medical devices.

In various aspects, a LAUD includes inflow and outflow cannulas for attachment to the left atrium 114 and the descending aorta 112 respectively. The inflow cannula provides the connection point between the LAUD and the left atrium 114, allowing blood to enter the device from the cardiac chamber. The outflow cannula establishes the connection between the LAUD and the descending aorta 112, directing blood from the device into arterial circulation. In some embodiments, each of the inflow cannula and the outflow cannula may have a diameter that ranges from between about 8 millimeters (mm) to about 12 mm, or from about 8.5 mm to about 11.5 mm, or from about 9 mm to about 11 mm, or from about 9.5 mm to about 10.5 mm, or about 10 mm. In some aspects, a length of the inner cannula can be adjusted according to the patient's anatomy, and a length of the outer cannula can be adjusted according to the patient's anatomy. The lengths of the inner cannula and the outer cannula may be the same or different from each other.

In some embodiments, a LAUD as described herein is a partial-support system composed of: a miniaturized centrifugal pump, an implantable battery, a bearing, a motor, inflow and outflow cannulas (e.g., as shown illustratively in FIGS. 1 and 2), an implantable controller, and a battery. Advantageously, the LAUD embodiments disclosed herein differs from prior devices in anatomy and physiology. For example, prior devices would draw from the left ventricle and return to the aorta, providing near-full replacement of left ventricle (LV) output (e.g., about 5 L/min) in series with the ventricle. Advantageously, a LAUD as disclosed in embodiments described herein can preserve native LV function and provide partial support by shunting blood from the left atrium to the descending aorta (e.g., at about 1.0 to about 2.5 L/min), operating in parallel with the ventricle. In various aspects, the effects are one or more of left-atrial pressure reduction, mitigation of pulmonary venous congestion, and augmentation of systemic flow without bypassing the native LV, which can advantageously correspond to the physiological needs or requirements of HFpEF patients. In some advantageous aspects, flow rate ranges of the various embodiments of the LAUD disclosed herein can provide sufficient left atrial decompression while avoiding the excessive flow rates.

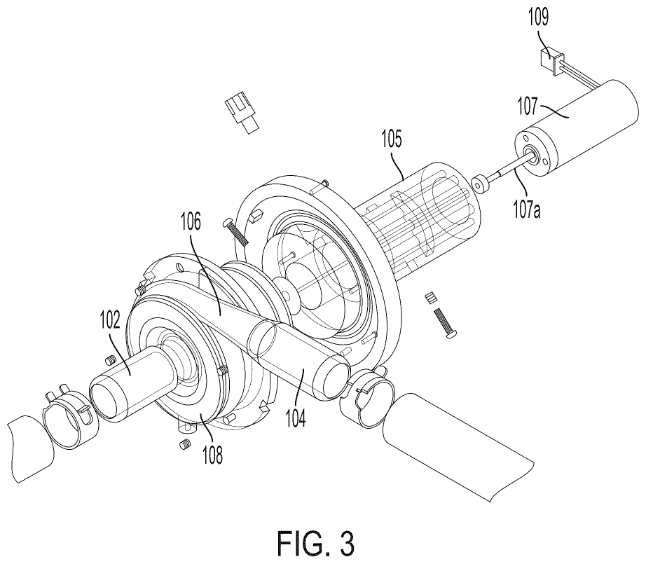

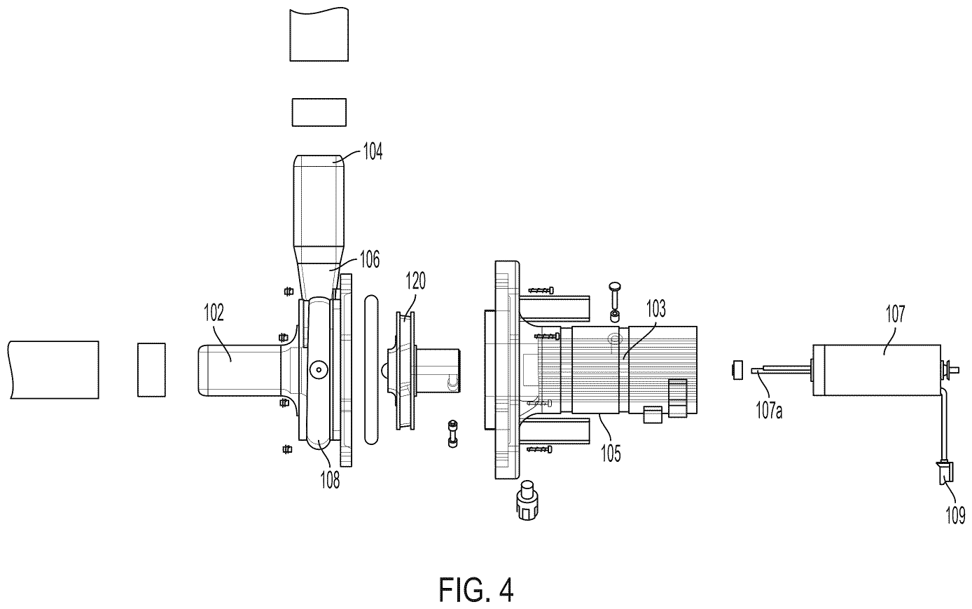

FIG. 3 shows an illustrative exploded perspective view of a LAUD, in accordance with various embodiments of the present disclosure. FIG. 4 shows an illustrative exploded side view of a LAUD, in accordance with various embodiments of the present disclosure. In various aspects, the LAUD uses a compact centrifugal pump, consisting of an inflow section 102, an outflow section 104, an impeller (not shown in FIG. 3) contained within a casing 108, and a volute 106. The inflow section 102 may be positioned at one end of the LAUD assembly and configured to provide an entry point for blood flow into the device. The inflow section 102 may be designed to receive blood from the left atrium 114 through various cannulation techniques. The inflow section 102 may include features that facilitate smooth blood entry and minimize turbulence as blood transitions from the cannula into the pump mechanism. The volute 106 may be positioned adjacent to the inflow section 102 and configured to house an impeller component within the LAUD assembly. The volute 106 may comprise a spiral-shaped chamber that facilitates the conversion of rotational energy from the impeller into fluid pressure and flow. The volute 106 may be designed with specific geometric parameters to optimize the conversion of rotational energy from the impeller 120 into fluid pressure and flow (e.g., to optimize hydraulic performance at targeted flow rates of about 1.0 to about 2.5 liters per minute). In various aspects, this advantageously reduces pressure in the left atrium.

The outflow section 104 may be positioned at an opposite end of the device from the inflow section 102. The outflow section 104 may be configured to direct blood flow from the volute 106 toward the descending aorta 112. The outflow section 104 may provide the exit pathway for blood that has been processed through the centrifugal pump mechanism, allowing for delivery of blood to the arterial circulation.

In the embodiments shown in FIGS. 3 and 4, a cover 105 encloses a motor 107. The motor 107 shown in FIGS. 3 and 4 is an illustrative component used for prototype testing and proof-of-concept evaluation; thus, the size and configuration of the motor 107 as depicted are not intended to limit the scope of the disclosure; in some embodiments, the motor may be smaller or otherwise dimensioned differently to be suitable for implantation in patients. The cover 105 in the embodiments shown in FIG. 3 was included to secure the motor 107 during prototype testing and may be omitted, modified, or integrated differently in various embodiments disclosed herein. Variations in motor size, mounting, and housing are not limited by the present disclosure. In some aspects, the motor 107 may be connected to a power supply (not shown) via a power supply connector 109. For example, a rotating shaft 107a may extend through the center of the volute 106 and may be configured to support and drive an impeller component. In some aspects, the rotating shaft 107a may be coupled to the impeller to transmit rotational motion from a drive mechanism to the impeller blades. The rotating shaft 107a may be supported by a bearing system (not shown) to enable smooth rotation during device operation.

In further embodiments, a motor may be operatively connected to a rotating shaft. The motor may provide the mechanical power necessary to rotate the impeller through the rotating shaft. In some embodiments, a power supply connector 109 may be attached to the motor to provide electrical power for device operation. In some aspects, casing 108 may enclose various assembled components of the LAUD. For example, the casing 108 may be configured to house the volute 106, a motor, and associated components.

The exploded view of FIGS. 3 and 4 demonstrate how various components may be arranged to form a LAUD in accordance with some embodiments disclosed herein. For example, in various embodiments, the inflow section 102 may connect to the volute 106, which houses the impeller mechanism driven by the rotating shaft. A motor may connect to a rotating shaft to provide rotational power, while the power supply connector 109 may supply electrical energy to the motor. In various aspects, the outflow section 104 may complete the fluid pathway by directing blood from the volute 106 toward the descending aorta 112.

Various components of embodiments of the LAUD described herein may be constructed from biocompatible materials suitable for long-term implantation in patients. Further, the illustrative components shown in FIGS. 3 and 4, are not shown to size and may be of comparatively different sizes or configurations than what is shown. For example, one or more of the components shown in FIGS. 3 and 4 may be smaller (e.g., the motor 107 and casing 108 may be a more compact size).

In various aspects, the LAUD comprises additional structural components including an impeller 120 and bearings (not shown in detail; an illustrative bearings location is shown at location 103) that provide rotational functionality for the pump assembly. The impeller 120 may comprise a central hub with multiple blades arranged to impart rotational energy to blood as the impeller 120 rotates within the casing 108. The impeller 120 may be configured to receive blood from the inflow section 102 and accelerate the blood outward through the action of the rotating blades.

The impeller 120 may include multiple blades that extend radially outward from a central hub region. In various aspects, the blades may be curved and positioned at specific angles to facilitate efficient fluid flow and pressure generation as the impeller 120 rotates. In some aspects, the blades may be straight and positioned at specific angles to facilitate efficient fluid flow and pressure generation as the impeller 120 rotates. The design of the impeller 120, including the number, shape, and arrangement of the blades, may be determined based on desired flow rates (e.g., flow rates of about 1.0 to about 2.5 liters per minute) suitable for HFpEF patients. In some embodiments, the LAUD includes an impeller and a volute configured to impart energy (e.g., to convert kinetic energy to static pressure) with improved efficiency and reduced losses. The impeller may include a hub, a shroud, and a plurality of blades extending radially outward from an inlet eye region toward an outlet periphery. Bearings may be configured to support the rotating shaft 107a and enable rotational movement of the impeller 120. The bearings may provide mechanical support for the rotating shaft 107awhile allowing smooth rotation during device operation. In various aspects, the bearings may be configured to minimize friction and wear during continuous operation of the LAUD. In some aspects, the bearings may comprise magnetic bearing systems that provide contactless support for the rotating shaft. Magnetic bearing systems may offer advantages in terms of reduced wear and decreased maintenance requirements. The magnetic bearing systems may utilize magnetic fields to support and position the rotating shaft without physical contact between bearing surfaces. For example, blood pumps may employ magnetic levitation (maglev) technology to suspend and rotate the impeller without mechanical contact. The bearings may be of any suitable available design or custom design and in various implementations, the bearings may interface with the designs and systems disclosed herein through standard mechanical structure. In some embodiments, magnetic bearings may reduce the risk of hemolysis by eliminating mechanical contact between moving parts. The bearings described herein may be modified or integrated differently in various embodiments. Variations in bearings (e.g., including location, shape, and size, among other properties) are not limited by the present disclosure.

In various embodiments, the volute 106 may be disposed adjacent the impeller 120 and may define a progressively expanding flow passage that receives fluid (e.g., blood) discharged from the impeller 120 and delivers the fluid towards the outflow section 104. The impeller 120 and volute 106 geometries may be dimensioned and proportioned to achieve advantageously uniform velocity distribution, reduced recirculation, and enhanced hydraulic efficiency across an intended operating range. The resulting configuration may provide improved pressure uniformity, reduced vibration, and increased durability for the LAUD, for example due to minimized cavitation and pressure pulsations. The motor 107 may be coupled to the rotating shaft 107a to provide mechanical power for driving the impeller 120. The motor 107 may transmit rotational motion through the rotating shaft 107a to the impeller 120, causing the impeller blades to rotate and move blood through the LAUD.

In various embodiments, the functional relationships between the components enable the LAUD to function to move blood from the left atrium 114 to the descending aorta 112. Blood may enter the device through the inflow section 102 and flow into the central region of the impeller 120. The bearings may enable continuous rotation of the rotating shaft 107a and impeller 120 while maintaining proper alignment and minimizing mechanical wear. As the impeller 120 rotates, driven by the motor through the rotating shaft 107a, the impeller blades may impart rotational energy to the blood, accelerating the blood outward toward the volute 106. The volute 106 may collect the blood discharged from the impeller 120 and convert the rotational energy into fluid pressure, directing the pressurized blood toward the outflow section 104. The outflow section 104 may then direct the blood through the outflow cannula 116 to the descending aorta 112, completing the blood flow pathway from the left atrium 114 to the arterial circulation. The illustrative configurations of components described herein may enable the LAUD to achieve advantageous flow rates (e.g., between about 1.0 and about 2.5 liters per minute), providing appropriate left atrial decompression for HFpEF patients. For example, the combination of the impeller 120, volute 106, bearings, and motor may advantageously create a compact LAUD that addresses the specific physiological requirements of patients with HFpEF.

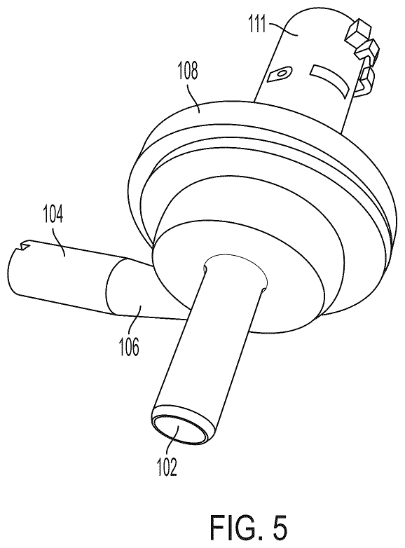

FIG. 5 shows an illustrative perspective view of a LAUD, in accordance with various embodiments of the present disclosure. In various aspects, FIG. 5 shows a LAUD having an inflow section 102, an outflow section 104, and a volute 106. In FIG. 5, the impeller (not shown in FIG. 5) is within the casing 108, and the rotating shaft (not shown in FIG. 5) is within the housing 111. The inflow section 102 may be positioned at one end of the LAUD and may provide the entry point for blood flow from the left atrium 114. The inflow section 102 may be integrated with the overall device structure to create a smooth transition for blood entering the pump mechanism. The inflow section 102 may be configured to minimize flow disturbances as blood transitions from the cannulation system into the pump assembly. The outflow section 104 may be positioned at an opposite end of the device from the inflow section 102 and may provide the exit pathway for blood flow toward the descending aorta 112. The outflow section 104 may be integrated into the overall device configuration to facilitate efficient blood discharge from the pump mechanism. The positioning of the outflow section 104 relative to the inflow section 102 may create a flow pathway that enables blood to traverse the pump assembly from the left atrium 114 to the descending aorta 112. The casing 108 may be configured to house the volute 106 and associated pump mechanisms. The volute 106 may be positioned within the casing 108 and may be integrated with the inflow section 102 and outflow section 104 to create a continuous flow pathway through the device.

A housing 111 may be positioned adjacent to the volute 106 and may contain additional components of the LAUD assembly. For example, the housing 111 may be configured to house components such as the motor (not shown in FIG. 5), bearings (not shown in FIG. 5), or other drive mechanisms that provide power for device operation. The housing 111 may be integrated with the casing 108 to form a unified structural assembly that encloses various components of the LAUD.

In various embodiments, the integrated design may provide a streamlined device profile that facilitates surgical implantation and positioning adjacent to the heart 132 and descending aorta 112. For example, the compact configuration may minimize the device footprint within the chest cavity 130 while maintaining the functional capabilities necessary to provide mechanical circulatory support for HFpEF patients. The assembled configuration may enable the LAUD to function as a mechanical circulatory support system that addresses the specific flow rate requirements of HFpEF patients. For example, in some aspects, the integration of the inflow section 102, volute 106, and outflow section 104 within the protective casing 108 and housing 111 may create a device that combines functionality with biocompatibility for long-term implantation applications.

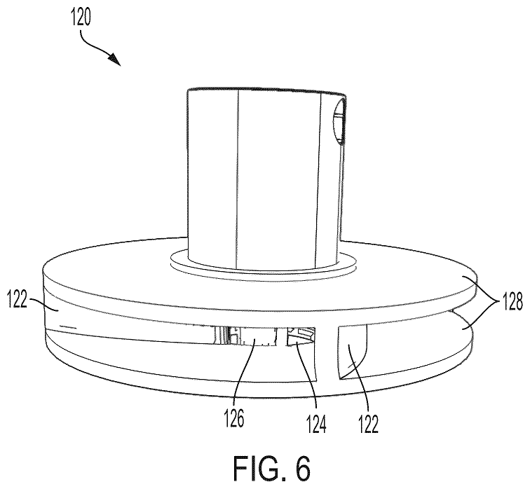

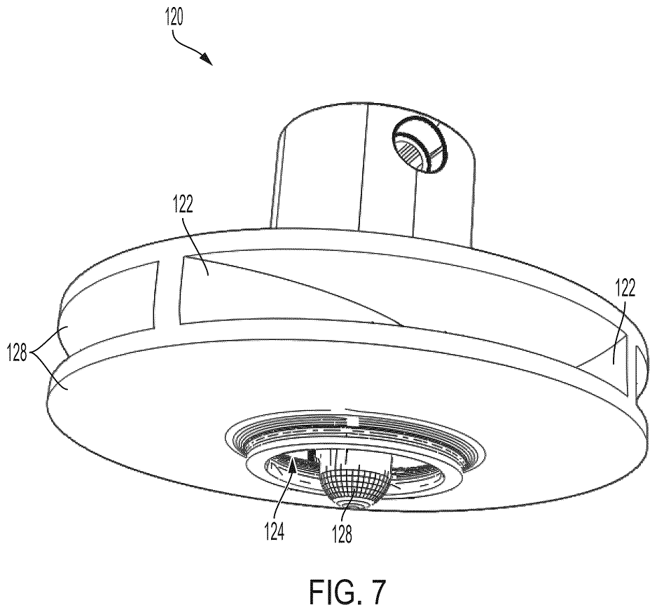

The size of the LAUD may be advantageous for its use. In various aspects, the LAUD may advantageously be configured with compact dimensions that facilitate implantation within the chest cavity 130 while maintaining the functional capabilities to transfer blood transfer from the left atrium 114 to the descending aorta 112 (as shown, for example, in FIGS. 1 and 2). In various aspects, the overall diameter of the impeller (e.g., which may be a diameter of the shroud(s)) may be about 3 centimeters (cm), which provides an exemplary reference scale for the compact configuration of the LAUD. In some embodiments, the diameter of the impeller may range from about 2.4 cm to about 3.6 cm, or from about 2.5 cm to about 3.5 cm, or from about 2.6 cm to about 3.4 cm, or from about 2.7 cm to about 3.3cm,, or from about 2.8 cm to about 3.2 cm, or from about 2.9 cm to about 3.1 cm, with corresponding proportional variations in width and height that maintain the advantageous size profile for implantation. For example, in various embodiments, a width of the impeller opening (for example, a distance between the shrouds (e.g., the shrouds 128 shown in FIG. 6), or a width of the blades) may range from about 2 mm to about 6 mm. In some aspects, the internal flow passage width of the impeller (for example, a width or diameter of the opening of the eye 124 as shown in FIG. 7) may range from about 2.5 mm to about 5.5 mm, or from about 3 mm to about 5 mm, or from about 3.5 mm to about 4.5 mm, or about 4 mm. The impeller diameter may be selected according to the desired head and flow rate, thereby producing a smooth flow transition at the outlet region. In various aspects, an interior width (e.g., an inner diameter) of the volute opening may be about 10 mm. In some aspects, an interior width of the volute opening may be about 8 mm to about 12 mm, or about 8.5 mm to about 11.5 mm, or about 9 mm to about 11 mm, or about 9.5 mm to about 10.5 mm.

The compact dimensions of the LAUD may provide several advantages for implantable applications in HFpEF patients. The smaller size may minimize may enable positioning of the LAUD within available anatomical spaces in the chest cavity 130 without requiring extensive tissue dissection or displacement of adjacent structures. The size characteristics of the LAUD may facilitate integration with the cardiovascular anatomy while maintaining the blood transfer function from the left atrium to the descending aorta. The compact dimensions may allow the device to advantageously be positioned in close proximity to the heart 132 and to attach to the left atrium and the descending aorta. The compact dimensions may advantageously minimize the length of required cannulation and reduce the complexity of the surgical implantation procedure. The compact dimensions may advantageously contribute to patient comfort and quality of life following implantation, as the compact device may create minimal anatomical disruption and may be less likely to interfere with normal chest wall movement or respiratory function. Various embodiments of the LAUD as described herein advantageously may provide the desired blood transfer capabilities (e.g., at flow rates of about 1.0 to about 2.5 liters per minute) while occupying minimal space within the chest cavity for HFpEF patients. These various embodiments are advantageously designed for the targeted patient population, with improved efficiency at the cardiac output range described herein (e.g., for blood flow rates of about 1.0 to about 2.5 liters per minute).

FIGS. 6 and 7 show views of an illustrative impeller in accordance with various embodiments disclosed herein. Referring to FIG. 6, the impeller 120 may comprise a structural configuration that enables efficient blood pumping within the LAUD assembly. The impeller 120 may include a hub 126, an eye 124, shroud(s) 128, and a plurality of blades 122 extending radially outward from the hub 126 region or the eye 124 region. The central hub may be configured to facilitate secure attachment to the rotating shaft 107a (shown in FIGS. 3 and 4), enabling transmission of rotational motion from the motor (illustratively shown in FIGS. 3 and 4) to the impeller 120.

As shown in FIG. 6, multiple blades 122 may extend radially outward from the central hub of the impeller 120 and in various embodiments may be connected to the shroud(s) 128. The blades 122 may be arranged in a circumferential pattern around the hub 126 (or the eye 124) in a configuration that provides balanced rotational characteristics during operation. In various aspects, the blades 122 may be configured to impart rotational energy to blood as the impeller 120 rotates within the volute 106 (shown in FIGS. 3-5), thereby accelerating blood from the central region toward the outer periphery of the impeller 120 to facilitate blood movement from the left atrium to the descending aorta.

In some embodiments, the blades 122 may exhibit straight or curved geometries that facilitate efficient fluid flow through the impeller 120, contributing to the advantageous blood pumping function that reduces left atrial pressure. For example, the size and/or curvature of the blades 122 may be designed to provide smoother blood movement from the inflow section 102 to the outflow section 104 of the impeller 120. In various aspects, the curved configuration of the blades 122 may decrease flow separation and turbulence, contributing to improved device functioning during pumping operation. In some embodiments, the blades 122 may be positioned at specific angles relative to the radial direction to optimize fluid flow characteristics. The blade angles may be selected to provide advantageous flow guidance and pressure generation as blood moves through the impeller 120.

In various embodiments, the impeller 120 may include an eye 124 located at the center of the impeller 120, forming a region (e.g., an inlet region at the eye 124) where blood enters the impeller 120 from the inflow section 102. The impeller eye 124 may function to provide a smooth transition for blood flowing from the inflow section 102 into the rotating impeller 120, facilitating the advantageous blood movement from the left atrium 114. In some aspects, the eye 124 minimizes flow disturbances as blood transitions from the stationary inflow section 102 to the rotating impeller 120. The eye 124 may include a single inlet aligned with the axis of rotation to admit fluid uniformly into the impeller passages. Alternatively, the eye 124 may include multiple inlets (e.g., dual inlets) positioned on opposite sides of the hub 126 to balance axial thrust and improve inlet flow symmetry. The eye 124 may be any shape, including conical, cylindrical, or rounded, and may include various features such as guide vanes or inlet diffusers. The diameter of the eye 124 may be varied relative to the hub 126 to affect parameters of the LAUD, such as desired inlet velocity. For example, the eye 124 may be configured with geometric parameters that facilitate efficient blood entry into the impeller 120 and contribute to the advantageous blood pumping function. In some aspects, the size and shape of the impeller eye 124 can provide adequate flow area while maintaining structural integrity of the impeller 120. The impeller eye 124 can thus, in various embodiments, serve as the primary inlet for blood flow, directing blood toward the blades 122 where rotational energy is imparted to the fluid.

FIG. 7 shows a further view of an illustrative impeller in accordance with various embodiments disclosed herein. For example, as shown in FIG. 7, the impeller 120 can include shrouds 128 that provide structural support and flow guidance for the blood pumping function. The shrouds 128 may function to contain the blood flow within passages created by the blades 122 and thereby direct blood toward the volute 106 (shown, for example, in FIGS. 3-5 and 9), facilitating the advantageous blood movement from the left atrium to the descending aorta. The shrouds 128 may comprise multiple shrouds (e.g., a front shroud that covers the inlet side of the blades 122 and a back shroud that covers the discharge side of the blades 122). Alternative shroud configurations may include partial shrouds that cover only portions of the blades 122, open impeller designs with only one shroud or no shrouds, or variable-thickness shrouds.

As shown in FIG. 7, the blades 122 may be arranged circumferentially around the impeller 120 to provide balanced fluid acceleration during rotation. The circumferential arrangement of the blades 122 may ensure uniform blood flow distribution as the impeller 120 rotates. The blades 122 may be configured to direct blood outward (e.g., toward the volute) for subsequent discharge through the outflow section.

In various embodiments, the volute 106 comprises an outer casing that defines the spiral-shaped chamber, creating a progressively expanding flow passage that collects blood discharged from the impeller 120. For example, the volute 106 can include an upper curved section that transitions into a discharge outlet region, providing a continuous flow pathway for blood moving from the impeller 120 toward the outflow section 104. In various aspects, the spiral configuration of the volute 106 may advantageously enable efficient conversion of the kinetic energy imparted by the rotating impeller 120 into static pressure, contributing to the blood pumping function that reduces left atrial pressure in HFpEF patients. In some embodiments, the volute 106 may have a wrap angle between about 270 degrees and about 360 degrees, a progressively increasing cross-sectional area to convert velocity to pressure, and a cutwater clearance (e.g., a height) of about 0 mm to about 6 mm, or about 1 mm to about 5 mm, or about 2 mm to about 4 mm, or about 3 mm, or between about three percent and about ten percent of the impeller outlet diameter, or between about 4 percent and about 9 percent of the impeller outlet diameter, or between about 5 percent and about 8 percent of the impeller outlet diameter, or between about 6 percent and about 7 percent of the impeller outlet diameter. The spiral start position of the volute (e.g., the specific angular position at which the spiral curve of the casing begins) may be between about 10° and about 30°, or between about 15° and about 25°, or between about 17° and about 23°, or between about 19° and about 21°, or about 20°. A throat area may be sized to match the flow rate while minimizing turbulence and pressure losses.

In various embodiments, the impeller geometry may include any number of blades (e.g., between four to six blades), and blade inlet and outlet angles may be determined based on desired parameters (e.g., to balance head and efficiency of the LAUD). In various aspects, parameters such as a blade wrap angle, hub-to-shroud ratio, and impeller outlet width may be defined to control velocity distribution and loading characteristics across the blade passages. In some aspects, the impeller 120 may comprise four blades arranged circumferentially around the central hub. For example, the four blades may be positioned at equal angular intervals of approximately 90 degrees around the central hub. In various aspects, the impeller 120 may comprise six blades arranged circumferentially around the central hub. For example, the six blades may be positioned at equal angular intervals of approximately 60 degrees around the central hub, and may provide more frequent fluid acceleration events during each rotation cycle. In various aspects, different blade angles may provide varying degrees of flow guidance and pressure generation, allowing the impeller 120 to be chosen for particular flow rate and pressure requirements that are suitable for HFpEF patients. The illustrative LAUDs as described herein may provide advantageous parameters such as flow rate, pressure generation, efficiency, and biocompatibility to address specific needs of HFpEF patients. For example, the various embodiments disclosed herein are advantageously designed for the targeted patient population, with improved efficiency at the cardiac output range described herein.

In various aspects, the energy and thermal profile may be optimized for implantable operation. At partial-support flows and modest head, expected electrical power draw may be about 2 to about 3 Watt (W) range under nominal physiology. The system may integrate transcutaneous energy management and transfer (TEMT) and an implantable battery for un-tethered operation; total losses (copper, core, switching, hydraulic) may be budgeted to keep tissue-facing temperatures less than 39° C. under continuous duty, with the controller throttling speed or charging rate if thermal headroom narrows.

An illustrative clinical intent of the LAUD may be to advantageously operate in parallel with the native left ventricle (LV), lowering left-atrial pressure, reducing pulmonary venous congestion, and augmenting systemic flow without bypassing ventricular ejection or abolishing aortic pulsatility. In some embodiments, typical use may target about 1.0 to about 2.0 L/min at rest with headroom to about 2.5 L/min during exertion or decompensation, adjustable to patient size and hemodynamics. Thus, the various embodiments may be advantageously designed for the targeted patient population, with improved efficiency at the cardiac output range described herein (e.g., for blood flow rates of about 1.0 to about 2.5 L/min).

In some embodiments, various reliability and safety features may be incorporated throughout the LAUD. For example, Maglev may eliminate bearing wear and lubricant paths; the pump may be purge-less, with smooth blood-contacting surfaces and minimized stagnant zones. Fault handling may include speed derate on low preload, over-temp throttling, charge-while-running management, and telemetry for trend-based thrombosis detection (e.g., rising torque at fixed flow), and early service interventions. Advantageously, acoustic and vibration signatures may be intrinsically lower due to the lower revolutions-per-minute (RPM) regime. Thus, in some aspects, the LAUD's centrifugal, low-RPM, maglev design may match the pressure-flow demands of left-atrial to aortic shunting while prioritizing hemocompatibility, thermal safety, and robust preload-sensitive control for durable, physiologically harmonious partial support.

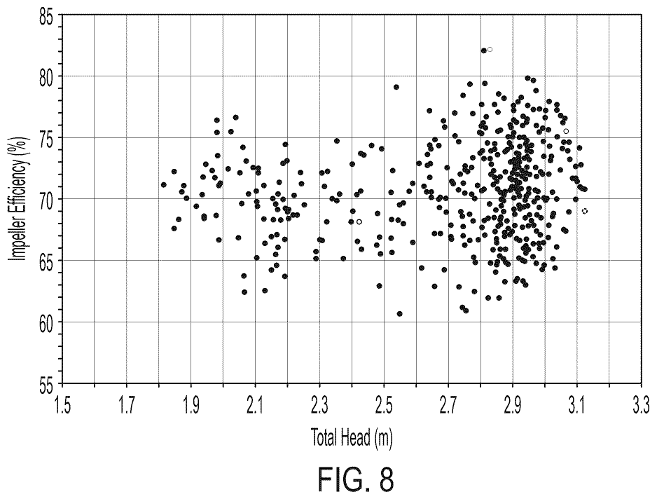

FIG. 8 shows an illustrative graph of impeller efficiency comparison for various designs of a LAUD, in accordance with various embodiments of the present disclosure. In some aspects, FIG. 8 illustrates a representative performance graph for some embodiments of LAUD designs disclosed herein. FIG. 8 illustratively plots impeller efficiency as a percentage (%) on the vertical axis as a function of total head in meters (m) on the horizontal axis. Total head is the total energy output of the LAUD as the equivalent height in meters of the pumped fluid (e.g., blood). Each point in FIG. 8 corresponds to a specific operating condition obtained either experimentally or through computational fluid dynamics (CFD) simulation, representing the hydraulic performance of the impeller across a range of flow rates. In some embodiments, the impeller geometry (for example, the blade curvature, shroud contour, and inlet and outlet radii may be modified to shift the impeller efficiency and total head. In various embodiments, the performance graph of FIG. 8 thereby serves as a verification and optimization tool for the impeller design.

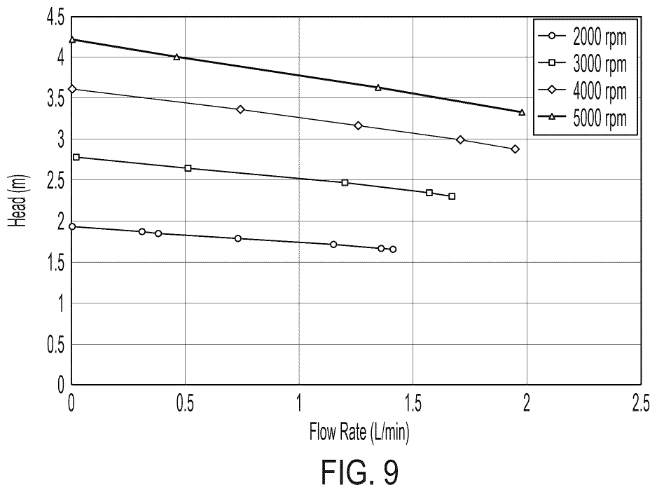

FIG. 9 shows an illustrative graph of head versus flow rate for various designs of a LAUD, in accordance with various embodiments of the present disclosure. In various aspects, FIG. 9 illustrates a representative performance characteristic graph of various embodiments of the impeller as disclosed herein, showing the relationship between total head (m) and flow rate (L/min) at various impeller rotational speeds. In this figure, the horizontal axis represents the flow rate, expressed in liters per minute (L/min), and the vertical axis represents the total head, expressed in meters (m).

In some embodiments, each plotted line corresponds to a different rotational speed of the impeller, for example 2000, 3000, 4000, and 5000 revolutions per minute (RPM), as shown in FIG. 9. In these illustrative embodiments, the plotted lines show a generally linear or near-linear relationship, where the total head increases with impeller speed and decreases with increasing flow rate. The linearity of the curves is characteristic of LAUD behavior under dynamically similar flow conditions, illustrating the fundamental scaling relationship between flow rate, head, and rotational speed for the LAUD.

In some embodiments, the shape or slope of the plotted lines (e.g., the head-flow rate curves) may be adjusted through modifications to the impeller blade geometry, such as changes to the outlet blade angle, the shroud contour, or the meridional flow passage. Such design modifications can allow tuning of the LAUD performance characteristics to achieve a desired balance between specific properties (e.g., flow rate, pressure generation, and efficiency for a given operating speed range). As discussed herein, durable mechanical circulatory support (MCS) devices have effectively improved survival and quality of life for patients with end-stage heart failure with reduced ejection fraction (HFrEF), typically generating flow rates of about 5 L/min or higher. However, these devices are not suitable for preserved ejection fraction (HFpEF) conditions, which require much lower flow rates. In other words, prior blood pumps exhibit low efficiency at low flow rates (e.g., 1-2.5 L/min). Accordingly, the LAUD, implanted within the chest cavity, connects the left atrium directly to the descending aorta and is designed to advantageously generate flow rates of up to about 2 L/min, thereby advantageously achieving higher efficiency within the desired flow range for HFpEF patients. These various embodiments are advantageously designed for the targeted patient population, with improved efficiency at the cardiac output range described herein (e.g., for blood flow rates of about 1.0 to about 2.5 liters per minute).

In some embodiments, the LAUD components, including impeller and volute geometries, are defined according to design parameters selected based on various parameters, such as size, desired head, and efficiency. For example, in various embodiments, the centrifugal impeller (e.g., impeller 120), compared with axial designs, may deliver the required pressure head at lower rotational speed (e.g., lower tip velocity), which may reduce shear exposure and hemolysis risk. The LAUD may be configured to provide a flow rate of about 1.0 to about 2.5 liters per minute (L/min), with a design head sufficient to overcome the left atrial-to-descending aortic pressure gradient ranging from about 50 to about 140 mmHg, which may advantageously accommodate hypotensive, normotensive, and hypertensive physiological conditions.

As used herein, the recitation of “at least one of A, B and C” is intended to mean “either A, B, C or any combination of A, B and C.” The previous description of the disclosed embodiments is provided to enable any person skilled in the art to make or use the present disclosure. Various modifications to these embodiments will be readily apparent to those skilled in the art, and the generic principles defined herein may be applied to other embodiments without departing from the spirit or scope of the disclosure. Thus, the present disclosure is not intended to be limited to the embodiments shown herein but is to be accorded the widest scope consistent with the principles and novel features disclosed herein.

The terms and expressions employed herein are used as terms and expressions of description and not of limitation, and there is no intention, in the use of such terms and expressions, of excluding any equivalents of the features shown and described or portions thereof. Each of the various elements disclosed herein may be achieved in a variety of manners. This disclosure should be understood to encompass each such variation, be it a variation of an embodiment of any apparatus embodiment, a method or process embodiment, or even merely a variation of any element of these. Particularly, it should be understood that the words for each element may be expressed by equivalent apparatus terms or method terms, even if only the function or result is the same. Such equivalent, broader, or even more generic terms should be considered to be encompassed in the description of each element or action. Such terms can be substituted where desired to make explicit the implicitly broad coverage to which this invention is entitled.

As but one example, it should be understood that all action may be expressed as a means for taking that action or as an element which causes that action. Similarly, each physical element disclosed should be understood to encompass a disclosure of the action which that physical element facilitates. Regarding this last aspect, by way of example only, the disclosure of a “protrusion” should be understood to encompass disclosure of the act of “protruding,” whether explicitly discussed or not, and, conversely, were there only disclosure of the act of “protruding,” such a disclosure should be understood to encompass disclosure of a “protrusion.” Such changes and alternative terms are to be understood to be explicitly included in the description.

What is claimed is:

1. A left atrium unloading device, comprising:
an inflow section configured to receive blood from a left atrium;
an impeller having multiple blades arranged move the blood;
a motor to rotate the impeller;
a volute; and
an outflow section configured to direct the blood from the volute to a descending aorta.

2. The device of claim 1, wherein the impeller comprises four blades.

3. The device of claim 1, wherein the impeller comprises six blades.

4. The device of claim 1, wherein the left atrium unloading device is configured to drain blood from a left atrium to a descending aorta at flow rates between about 1.0 and about 2.5 liters per minute.

5. The device of claim 1, wherein the impeller comprises an impeller eye located at a center of the impeller forming an inlet region where blood enters the impeller.

6. The device of claim 1, wherein the volute comprises a spiral-shaped chamber configured to convert rotational energy into fluid pressure.

7. The device of claim 1, further comprising bearings supporting a rotating shaft.

8. The device of claim 7, wherein the bearings comprise magnetic bearing systems.

9. The device of claim 1, further comprising a power supply connector attached to the motor for electrical power delivery.

10. The device of claim 1, further comprising an inflow cannula attached to the left atrium and an outflow cannula attached to the descending aorta.

11. A method of treating heart failure with preserved ejection fraction, comprising:
implanting a left atrium unloading device within a chest cavity of a patient, the left atrium unloading device comprising:
an impeller;
an inflow section configured to receive blood from a left atrium of the patient; and
an outflow section configured to transfer blood to a descending aorta of the patient,
wherein the left atrium unloading device is configured to rotate the impeller to remove blood from the left atrium to the descending aorta.

12. The method of claim 11, wherein the impeller comprises four blades.

13. The method of claim 11, wherein the impeller comprises six blades.

14. The method of claim 12, wherein the blades are curved.

15. The method of claim 11, wherein implanting the left atrium unloading device comprises positioning the device in the patient through a thoracotomy procedure.

16. The method of claim 11, wherein implanting the left atrium unloading device comprises positioning the device in the patient through a sternotomy procedure.

17. A left atrium unloading device for heart failure with preserved ejection fraction patients, comprising:
an inflow section configured to receive blood from a left atrium;
an impeller having multiple blades arranged move the blood;
a motor to rotate the impeller;
a volute; and
an outflow section configured to direct the blood from the volute to a descending aorta.

18. The device of claim 17, wherein the left atrium unloading device is configured to drain blood from a left atrium to a descending aorta at flow rates between about 1.0 and about 2.5 liters per minute.

19. The device of claim 17, further comprising a power supply connector attached to the motor for electrical power delivery.

20. The device of claim 17, further comprising an inflow cannula attached to the left atrium and an outflow cannula attached to the descending aorta.
